(19) United States
(12) Patent Application Publication
(10) Pub. No.: US 2017/0360653 A1
(43) Pub. Date: Dec. 21, 2017

(54) MALE SEXUAL STIMULATION DEVICE

(71) Applicant: Ian Peter Marshall, Kettering (GB)

(72) Inventor: Ian Peter Marshall, Kettering (GB)

(21) Appl. No.: 15/186,566

(22) Filed: Jun. 20, 2016

(51) Int. Cl. A61H 21/00; A61H 23/00; A61F 5/41; A61H 19/00; A61N 1/00

(52) U.S. Cl. CPC A61H 21/00; A61H 19/32; A61N 1/00; A61F 5/41; A61H 23/00; A61H 2201/169; A61H 2201/0107

(57) ABSTRACT

A male sexual stimulation device comprising: an anal or prostate stimulation shaft; and a holder for holding a male penile stimulation device, wherein said anal stimulation shaft is connected to the holder by a connecting strap, and wherein the holder of the male penile stimulation device is in the form of one or more closed rings/loops or adjustable straps. The function of the holder connected the anal or prostate stimulation shaft is to hold any conventional male penile masturbation device firmly, so that the male sexual stimulation device disclosed according to the invention, along with any conventional male penile masturbation device, can function as an apparatus to provide simultaneous sexual stimulation to more than one erogenous zones such as the penis, perineum, anus and the P-spot.

### FIELD OF THE INVENTION

The present invention pertains to sexual stimulation devices in general and, more particularly, to an anal or prostate stimulation device capable of holding hand-held male penile masturbation devices which are generally elongated.

### BACKGROUND ART

There are many sexual stimulation zones in/on a body of a human male. Physical stimulation of more than one erogenous zone at the same time provides enhanced sexual stimulation to men. Simultaneous massaging of the penis, perineum, anus, rectum, and the P-spot produces synergistic or enhanced effect of sexual stimulation in men.

The perineum, which is a highly innervated and erogenous zone, is an important part of the male sexual stimulation cycle/response. It is a proven fact that massage of the male perineum can be very pleasing and soothing. Applying pressure on the perineum provides therapeutic benefits as well. Applying pressure and massaging the acupressure point on the perineum helps in treatment various ailments or conditions such as constipation, impotence, hemorrhoids, and rectal prolapse.

The prostate gland is also an integral part of the male sexual response cycle. Due to its proximity to the anterior rectal wall, the prostate gland can be massaged from the anterior wall of the rectum. It is worth noting that there is a wall of tissue between the rectum and the prostate gland. The area in the anterior wall of the rectum where the prostate gland can be massaged indirectly is widely known as “Prostate-spot” or “P-spot”. The P-spot is located approximately 2 inch (5.08 cm) inside the rectum in the anterior wall of the rectum. Massaging of the prostate gland is done by rubbing or stroking the P-spot in the anterior rectal wall. As the P-spot is located anatomically very near to the internal root of the penis, massaging of the prostate gland also stimulates the penis. Massaging the prostate is very pleasurable when it is done with physical stimulation of the penis. It is experienced by many men that prostate massage also helps in triggering an intense orgasm. The prostate gland secretes fluid called “prostate fluid” that nourishes and protects sperm. During ejaculation at orgasm, the prostate fluid is expelled with sperm as semen. Massaging of the P-spot can also increase the quantity of seminal fluid which increases the survival chances of sperm in the vagina or sperm storage devices. The P-spot can also be massaged and stimulated from outside, usually by rubbing and applying pressure on the perineum. Therefore, massaging the perineum with pressure on it serves two purposes—perineum massage and prostate massage.

There are butt plugs/anal plugs and prostate massaging devices available in the market for massaging the anus and the P-spot, but none of them is designed for massaging/stroking the penis as well at the same time. There are also butt plugs and prostate massagers having a penis ring or/and a ball ring attached to it with the help of a connecting strap; however, the penis rings or the ball rings attached to such butt plugs and prostate massagers are not designed to enable palm or hand to massage or stroke an erect penis for sexual stimulation. Rather, the penis rings attached such butt plugs or prostate massagers are meant to be placed around at the base of the penis to maintain erection of the penis by slowing down the backflow of blood from the erect penile tissue. Butt plugs and prostate massagers having penis ring are all designed to be fixed or immovable once worn or put on. The internal diameter of such penis rings attached to butt plugs and prostate massagers is generally 30 mm to 40 mm, which is not large enough to accommodate any conventional male penile masturbation devices.

Conventional hand-held male penile masturbation devices in prior art are generally elongated or/and cylindrical. These hand-held penile masturbation devices generally have an opening/orifice for inserting the penis through the central hollow space along the length. One of the examples is the device sold with the trademark “Fleshlight”. All these male penile masturbation devices are designed to run up and down along the length of an erect penis. In other words, these conventional male masturbation devices are designed for stroking the penis only. None of them is capable of massaging the perineum, anus, and the P-spot as well at the same time. It is worth noting that the anus is rich with sensitive nerve endings.

If a man wants to massage the penis, anus and the P-spot at the same time, he will need to have at least two devices, namely a penile masturbation device and a butt plug/prostate massager. One of the disadvantages of using two different unconnected devices at the same time is that at least two hands are required to be employed at the same time to operate or/and move both the devices, which is a very cumbersome and distracting act during solo sexual stimulation. So, if a man wants to massage the penis, anus and the P-spot at the same time with one hand only, it would be necessary to have an apparatus or a device wherein two components (a penile masturbation device and a butt plug/prostate massager) are or can be physically connected to each other so that movement/motion of one of the components produces direct and corresponding movement or motion to the other or vice versa. One of the ways of connecting the devices is to have a suitable holder connected to the butt plug or prostate massager for holding the male penile masturbation devices. Another way is to have a holder attached to the male penile masturbation device for holding the butt plugs or prostate massagers.

In DE 4129177 C1, a device called “Suspensory” which has a penis ring connected to an anal/butt plug is disclosed. This device is mainly designed for curing erectile dysfunction of men. It does not have the features of a hand-held penile massager which can be used to stroke penis for masturbation.

The objective of the present invention is to provide an anal or prostate stimulation device having a means for holding a male penile masturbation device. When the anal or prostate stimulation device is connected firmly to a male penile masturbation device, simple stroking motion of the penile masturbation device will produce rhythmic or alternating motion or movement to the anal or prostate stimulation device as well. In this way, more than one erogenous zone (the penis, perineum, anus and the P-spot) can be massaged and stimulated at the same time.

### SUMMARY OF THE INVENTION

Subject matter of this invention is an anal or prostate stimulation device which can be physically connected to any conventional male penile masturbation devices which are usually elongated. A male sexual stimulation device in accordance with one embodiment of the invention comprises an anal or prostate stimulation shaft and a holder for holding male penile stimulation devices which are generally elongated, wherein said anal shaft is connected to the holder by a connecting strap, and wherein the holder of the male penile stimulation devices is in the form of one or more closed loops/rings or adjustable straps. The function of the holder, which may be in the form of a closed ring or an adjustable strap, connected the anal or prostate stimulation shaft is to hold any conventional male penile masturbation device firmly, so that the male sexual stimulation device disclosed according to the invention, along with any conventional male penile masturbation device, can function as an apparatus for achieving one or more objectives. The main objective of the apparatus is to enable movement/motion of the male penile masturbation device during stroking (masturbation) of the penis to produce corresponding movement/motion to the anal or prostate stimulation shaft as well. It is obvious that any movement of the anal or prostate stimulation will also produce corresponding movement/motion to the penile masturbation device as both the components are connected to each other.

There are many advantages of the invention. One of the main advantages of the invention is that the device disclosed according to the invention can provide physical sexual stimulation to more than one erogenous zones (the penis, perineum, anus and the P-spot) in men to produce a synergistic or an enhanced effect of sexual pleasure.

A further advantage of the invention is that only one hand needs to be employed for the effective operation of the device and the male penile masturbation device held by it. As both the anal shaft and the penile sexual stimulation device are connected by a strap, movement of either of the two produces movement or motion to the other.

A further advantage of the invention is that the device disclosed according to the invention can hold or accommodate any conventional male penile masturbation device of any design or size. Also the holder, being an adjustable or flexible one, gives the option of using more than one male penile masturbation devices at different time. More advantages of the present invention will be clearly evident from the drawings and description herein.

### BRIEF DESCRIPTION OF THE DRAWINGS

The invention will be more clearly understood from the following description of some embodiments thereof, given by way of example only, with reference to the accompanying drawings, in which:

FIG. 1 is a perspective view of a male sexual stimulation device according to one embodiment of the invention, wherein the male sexual stimulation device is provided with a closed elastic/flexible ring or loop for holding a male penile masturbation device;

FIG. 2 is a perspective view of a male sexual stimulation device according to one embodiment of the invention, wherein the male sexual stimulation device is provided with one adjustable strap for holding a male penile masturbation device;

FIG. 3 is a perspective view of a male sexual stimulation device according to one embodiment of the invention, wherein the male sexual stimulation device is provided with two adjustable straps for holding a male penile masturbation device;

FIG. 4 is a perspective view of an elongated male penile masturbation device having a regular smooth surface;

FIG. 5 is a perspective view of an elongated male penile masturbation device having more than one circular recess on its surface;

FIG. 6 shows an illustration of how the device in FIG. 3 holds the elongated male penile masturbation device shown in FIG. 4;

FIG. 7 shows an illustration of how the device in FIG. 1 holds the elongated male penile masturbation device in FIG. 5; and

FIG. 8 shows an illustration of how the apparatus shown in FIG. 6 is worn for use, showing how the device 3 works.

### DETAILED DESCRIPTION OF THE INVENTION

Referring initially to FIG. 1 in particular, there is shown a male sexual stimulation device 1 in accordance with one embodiment of the invention, comprising: an anal or prostate stimulation shaft 1a and a closed elastic or flexible holder loop/ring 6 connected to each other by a connecting strap 7 which may be rigid or resiliently flexible or elastic. For the purpose of description of this invention, the anal or prostate stimulation shaft 1a may also be referred to as “anal plug” or “butt plug” or “prostate massager”, all of which mean one and the same thing. Preferably, the anal plug 1a is configured to touch the P-spot when inserted in the rectum. The anal plug 1a is dimensioned to be inserted into the rectum, and is preferably of 2-4 inch (5.08-10.16 cm) in length. If the anal plug 1a is not long enough to touch the P-spot, then the device 1 will be capable of stimulating mainly the penis, the perineum and the anus, which is also one of the objectives of the invention. The anal plug 1a has a constriction 9, which preferably has a maximum cross sectional diameter of 2 inch (5.08 cm), adjacent to the base. The butt plug 1a, including the constriction 9, may be rigid or resiliently flexible. The anal plug 1a has a bulge (or a bulbous portion) 10 adjacent to the constriction 9 and towards the tip 11. The bulge 10 has a girth larger than the girth of the constriction 9. Preferably, the cross sectional diameter at the broadest point of the bulge 10 is generally between 0.5 inch (1.27 cm) to 3 inch (7.62 cm). In a fully worn or inserted state of the anal plug 1a, the constriction 9 is supposed to be surrounded and gripped by the anal ring or the anal sphincter. Once inserted in the rectum, the bulge 10 will stop the butt plug 1a from coming off the rectum easily. The butt plug 1a may have different designs; however, at least a bulge or bulbous portion having at least 0.5 inch (1.27 cm) cross-sectional diameter is desirable at a position adjacent to constriction 9. In case there is no bulge or bulbous part like bulge 10, it is desirable that the area right above the constriction 9 has a greater diameter than the diameter of the constriction 9, so that the area of greater diameter above the constriction 9 will stop the anal plug 1a from slipping out of the rectum easily. If the anal plug 1a does not have any constriction like the constriction 9 or/and any bulge or bulbous portion adjacent to it, the user should use one of his hands to stop the anal plug 1a from coming off the rectum. Also if the anal plug 1a has no constriction or/and bulge or bulbous portion along its shaft, a user can stop the anal plug 1a from coming off the rectum by sitting on the anal plug 1a after the same has been inserted and placed in the rectum, although it may not be very comfortable for many men. In a preferred embodiment, the distal end/tip 11 of the anal plug 1a is curled or bent towards the connecting strap 7. Preferably, the curled or bent tip 11 is located between 5.08 cm and 10.16 cm away from the constriction 9. The curled tip 11 will press or rub against the P-spot during the operation of the device 1. In an alternative embodiment, the butt plug 1a may not have a curled tip, although it is not going to be as effective as the curled or bent one for massaging of the p-spot. Alternatively, there may be more than one regular or irregular bulge/bulbous portion along the length of the anal plug 1a. The anal plug 1a may also have one or more bumps or protrusions or the like facing the strap 7 for touching the P-spot during the use of the device 1. In a preferred embodiment, the connecting strap 7 has a nub or bulge 8 on its upper surface near or adjacent to the base of the anal plug 1a. The nub 8 may be provided with nodules/ridges on it. This nub 8 has the function of applying pressure on the perineum during the operation of the device 1. At the distal end of the strap 7 is a closed flexible or elastic holder 6 which has the function of holding any hand-held conventional male penile masturbation devices. The strap 7 has at least the minimum length or elasticity or flexibility required to cover the distance between the anus and base of the penis above the scrotum. In a preferred embodiment, if the strap 7 is not flexible or elastic, the strap 7 is at least 2 inch (5.08 cm) in length. Different versions of device 1 having strap 7 of different lengths or flexibilities may be manufactured to suit different men having different lengths of the perineum and the penis. Also if one wants to shorten the strap 7, making a knot out of the strap 7 might work. The strap 7 may be flexible (resiliently or otherwise), elastic or stretchable. The flexibility or stretchability can be achieved by having appropriate mechanical means or suitable material. The holder ring/loop 6 has a diameter or/and elasticity or flexibility suitable for holding conventional male penile masturbation devices firmly. Conventional penile masturbation devices in prior art are generally elongated. However, any hand-held male penile masturbation devices which may not be elongated can also be held by the device disclosed according to the present invention. In an alternative embodiment, more than one closed loop 6 may be provided along the length of the strap 7. Considering the flexibility of the strap 7, the loop 6 may lie in any plane. In an alternative embodiment, the strap 7 is detachable from the anal or prostate stimulation shaft, wherein attaching or holding or fastening means, such as a simple elastic or flexible closed loop or ring or adjustable strap(s), may be provided at the proximal end of the strap 7.

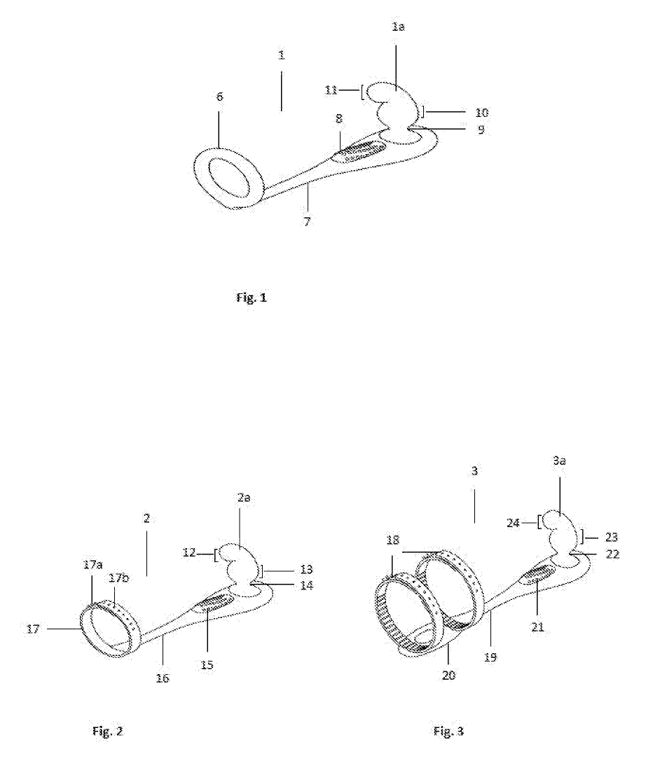

FIG. 2 shows an alternative embodiment of the invention, wherein the closed loop 6 in the device 1 is replaced with an adjustable (closable and openable) strap 17 for holding a male penile masturbation device. The device 1 in the FIG. 1 and the device 2 in the FIG. 2 have the same features/structures except the difference in the feature(s) of the holders (6, 17), meaning thereby the description of the anal plug 1a and the strap 7 above in case of the device 1 applies to the anal plug 2a and strap 16 respectively. To a person skilled in the art, this difference will be evident from the figures provided herewith. Instead of a closed and flexible/elastic holder 6 in the device 1, the holder 17 in the device 2 in the FIG. 2 is an adjustable strap having stud 17a and holes 17b for closing or locking the strap 17. Alternative locking/closing mechanisms/means may be employed, such as magnetic locking, buckle locking, Velcro, etc. This feature of the strap 17 being an adjustable one (openable and closable one) makes the device 2 practically suitable for holding any conventional male penile masturbation devices of any size/circumference.

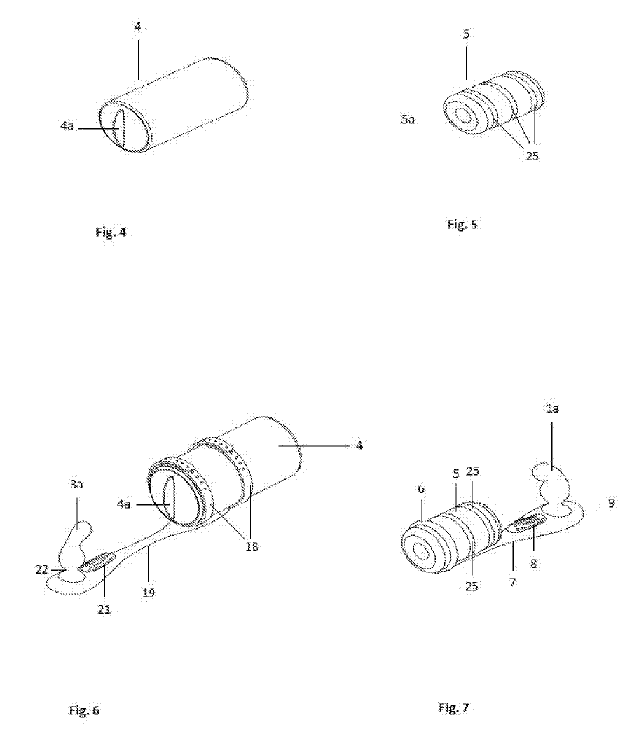

FIG. 3 shows an alternative embodiment of the invention. Instead of one adjustable strap holder 17 in the device 2, two adjustable straps 18 are provided for holding a male penile masturbation device. The device 2 in the FIG. 2 and the device 3 in the FIG. 3 have the same features except the fact that instead of one adjustable holding strap 17 in the device 2, there are two holding straps 18 in the device 3. The description of the anal plug 1a and the strap 7 above in case of the device 1 applies to the anal plug 3a and strap 19 respectively. In accordance with the current invention, one or more closed or adjustable straps or ring may be employed for holding a conventional male penile masturbation device. Employing more than one closed or adjustable holder may provide more firmness in holding a male penile stimulation device.

FIG. 4 is a perspective view of an elongated male penile masturbation device 4 having a regular smooth surface, which is given by way of example only. FIG. 5 is a perspective view of an elongated male penile masturbation device 5 having 3 circular recesses 25 on its surface, which is also given by way of example only. The circular recesses 25 are designed to engage with the circular elastic or flexible ring 6 to form the usual male and female interaction so that the male penile stimulation device 5 is held firmly by the holders 6. The circular recesses 25 minimize the chances of holder ring 6 slipping over the surface of the male masturbation device 5.

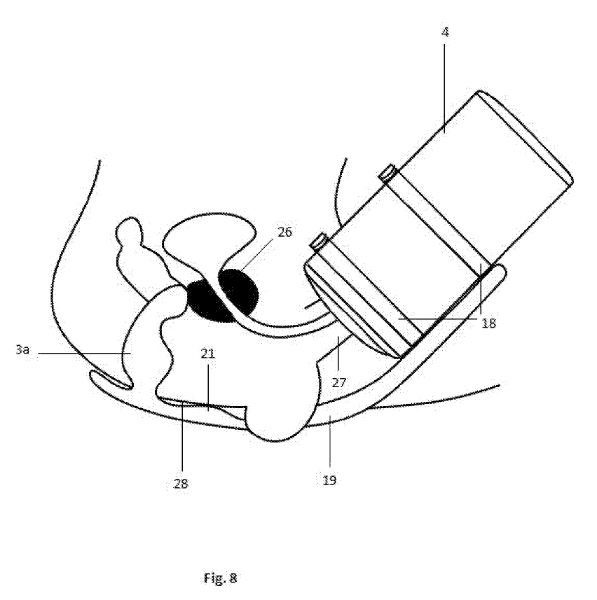

FIG. 6 shows an illustration of how the device 3 in FIG. 3 holds the elongated male penile masturbation device 4 in FIG. 4. This is just an example only. Any conventional male penile sexual stimulation device of any design may be technically employed in place of the device 4. The adjustable straps 18 can hold the device 4. When fully assembled as an apparatus ready for use, the penis insertion opening/orifice 4a faces the anal plug 3a.

FIG. 7 shows an illustration of how the device 1 in FIG. 1 holds the elongated male penile masturbation device 5 in FIG. 5. The loop 6 will fall in one of the recesses 25 to form male-female interaction. When fully assembled for use, the penis insertion opening or orifice 5a faces the anal plug 1a. Having multiple recesses 25 on the device 5 allows a person to adjust the distance between the penile opening 5a and the anal plug 1a. This is just an example only. Any conventional male penile sexual stimulation device of any design may be technically employed in place of the device 5.

FIG. 8 shows an illustration of how the assembled apparatus in FIG. 6 is worn and ready for use. During the practical use of the apparatus in FIG. 6, the anal plug 3a of device 3 is fully placed in the rectum, making sure that the anal sphincter or anal ring surrounds and grips the constriction 22. The bulge 23 goes beyond the anal sphincter or anal ring into the rectum. Placement of bulge 23 in this way in the rectum makes sure that the butt plug 3a does not come off the rectum easily during the intermittent/rhythmic pull of the strap 19 towards the device 4 during the use of the device 3. As described above, there may be more than one bulge/bulbous portion along the length of the anal plug 3a. The constriction (a circular groove or recess) at the base of the anal plug 3a in all the embodiments of the invention discussed above preferably has at least a length of 0.5 inch (1.27 cm). The penis 27 is inserted into the penile stimulation device 4 through the orifice 4a. The adjustable strap 18 will hold the device 4 firmly. Instead of adjustable strap 18 having stud and holes for closing or locking or fastening the strap 18, alternative locking or closing or fastening mechanisms/means may be employed, such as magnetic locking, buckle locking, Velcro, etc. In case of the device 2, only one strap 17 will hold any male penile masturbation devices; whereas, in case of the device 1, only one closed flexible/elastic rings 6 will hold any male penile masturbation devices. When the device 3 is fully worn, the strap 19 will run over the perineum 28 of the user. The strap 19, if rigid, has suitable length to cover at least the length of the perineum 28 or the distance between the anus and base of the penis 27. The nub 21 will press against the acupressure point or surrounding area on the perineum 28. When the erect penis 27 is placed in the penile massager 4 with the strap 19 running over the perineum, the curled tip 24 of the butt plug 3a in the rectum will touch the P-spot or area around it on the anterior wall of the rectum. When the user grips the penile massager 4 and moves it (strokes it) over the erect penis 27 up and down repeatedly/rhythmically, this motion/movement of stroking produces direct and corresponding pull and release of the strap 19, which will, in turn, directly produces movement/motion to the anal plug 3a including the curled tip 24 in the rectum. When the strap 19 is pulled and released alternatively or rhythmically during penile masturbation by the device 4 in this way, the curled tip 24 will provide varying pressure on the P-spot or/and the surrounding area. This application of varying pressure on the P-spot or surrounding area by the curled tip 24 will massage and stimulate the prostate gland during the operation of the device 3. As the anal plug 3a is connected firmly to the penile massager 4, the curled tip 24 automatically remains facing the P-spot during the use of the device 3. Any bump or protrusion on the anal plug 3a capable of touching the P-spot can serve the purpose of the curled tip 24. When the strap 19 is pulled and released alternatively/rhythmically during penile masturbation, the nub 21 will press against the perineum 28 and slide over it up and down repeatedly. The massaging of the perineum 28 in this way will be more profound when the angle between the penis 27/device 4 and the abdomen is 45 degree (0.25π rad) or less during the use of the device 3. The simple motion of the penile massager 4 will produce synchronous movement/motion of all the main constituent parts (anal plug 3a, nub 21, and strap 19) of the device 3, which will provide simultaneous physical stimulation to the anus, rectum, P-spot, perineum, and the penis. Initial discomfort, if any, of the anal penetration by the anal plug 3a can be reduced drastically by simultaneous stimulation of other erogenous zones (the perineum, Penis, and the anus) by the devices 3 and device 4. The overall sexual stimulation produced by the device 3 and massager 4 will be more than the sexual stimulation produced by the constituent parts individually or separately. The anal plug 3a, penile massager 4 and nub 21/Strap 19 may be provided with means for vibration, actuation, thermal heating or electro-stimulation. There may be more than one control button to separately or concurrently control vibration or actuation or thermal heating or electro-stimulation in the anal plug 3a, penile massager 4 and nub 21/Strap 19, which will give the user the option for synchronous or asynchronous vibration/actuation/heating/electro-stimulation of the erogenous zones.

Similarly, the devices 1 and 2 will also function in the same way as the device 3, except the fact that a closed holder ring 6 and single adjustable strap 17 hold the devices 1 and 2 respectively. The only difference between the device 3 and the devices 1 and 2 is how the holders (6, 17, and 18) work to hold male penile masturbation devices. Adjustable holder straps 17 and 18 can accommodate and hold male penile masturbation devices of any size or/and design. One of the important aspects of the invention is that one or more holders (6, 17 and 18) may be employed to hold any male penile masturbation devices.

### Industrial Applicability

The device disclosed according to the invention can be used with any male penile masturbation devices in the market. The adjustability or flexibility of the holder attached to the device can accommodate any male penile masturbation device of any design or/and size. So, this gives a person the option to enjoy more than one male penile masturbation devices at different time.

The title of the invention does not limit the scope of claim in this application. Although some of the preferred embodiments of the present invention have been described herein, those skilled in the art will appreciate that various modification, additions and substitutions are possible without departing from the scope and spirit of the invention as disclosed in the accompanying claims.

What is claimed is:

1. A male sexual stimulation device comprising:
(i) an anal or prostate stimulation shaft dimensioned for insertion through the anus; and
(ii) a holder in the form of one or more closed rings or adjustable straps for holding a male penile sexual stimulation device;
wherein the anal stimulation shaft is connected to the holder by at least a connecting strap.

2. The male sexual stimulation device as claimed in claim 1, wherein the anal or prostate stimulation shaft has a constriction adjacent to its base.

3. The male sexual stimulation device as claimed in claim 2, wherein the constriction has a maximum cross sectional diameter of 3 inch (7.62 cm).

4. The male sexual stimulation device as claimed in claim 1, wherein the tip or the distal end of the anal or prostate stimulation shaft is curled or bent towards the connecting strap.

5. The male sexual stimulation device as claimed in claim 1, wherein the anal or prostate stimulation shaft has at least a bulge or a bulbous part adjacent to the constriction.

6. The male sexual stimulation device as claimed in claim 5, wherein the minimum cross sectional diameter of the bulge or the bulbous part of the anal shaft is at least 0.5 inch (1.27 cm) at its broadest point.

7. The male sexual stimulation device as claimed in claim 1, wherein the anal shaft has at least one or more bumps or protrusions or the like facing the connecting strap.

8. The male sexual stimulation device as claimed in claim 1, wherein the connecting strap has one or more nubs or bulges on its upper surface near or adjacent to the base of the anal or prostate stimulation shaft.

9. The male sexual stimulation device as claimed in claim 1, wherein the connecting strap is either elastic/flexible or rigid.

10. The male sexual stimulation device as claimed in claim 1, wherein the length of the connecting strap at its relaxed or stretched state is adequate to cover at least the length the perineum.

11. The male sexual stimulation device as claimed in claim 1, wherein the holder is in the form of one or more closed and flexible or elastic rings or loops.

12. The male sexual stimulation device as claimed in claim 1, wherein the holder is in the form of one or more adjustable straps with appropriate means to make it openable and closable.

13. The male sexual stimulation device as claimed in claim 1, wherein means for vibration or actuation or thermal heating or electro-stimulation is/are provided to one or more parts of the device.

14. The male sexual stimulation device as claimed in claim 1, wherein the connecting strap is detachable from the anal stimulation shaft.

15. An male sexual stimulation apparatus comprising:
(i) a male penile masturbation device;
(ii) an anal or prostate stimulation device; and
(iii) at least one connecting strap for connecting the male penile masturbation device and the anal or prostate stimulation device.

16. The male sexual stimulation apparatus as claimed in claim 15, wherein the male penile masturbation device is dimensioned to be held by one or both hands.

17. The male sexual stimulation apparatus as claimed in claim 15, wherein the male penile masturbation device has at least an opening or orifice for insertion of the penis at one end.

18. The male sexual stimulation apparatus as claimed in claim 15, wherein the male penile masturbation device has at least one circular recess perpendicular to its length on its surface.

19. The male sexual stimulation apparatus as claimed in claim 15, wherein the anal or prostate stimulation device is dimensioned for insertion through the anus.

20. The male sexual stimulation apparatus as claimed in claims 19, wherein the anal or prostate stimulation device has a constriction at its base.

21. The male sexual stimulation apparatus as claimed in claim 20, wherein the constriction has a maximum cross sectional diameter of 3 inch (7.62 cm).

22. The male sexual stimulation apparatus as claimed in claim 15, wherein the tip or the distal end of the anal or prostate stimulation device is curled or bent towards the connecting strap.

23. The male sexual stimulation apparatus as claimed in claim 19, wherein the anal or prostate stimulation device has at least a bulbous portion above the constriction.

24. The male sexual stimulation apparatus as claimed in claim 23, wherein the bulbous portion of the anal or prostate stimulation device has a minimum cross sectional diameter of 0.5 inch (1.27 cm) at its broadest point.

25. The male sexual stimulation apparatus as claimed in claim 15, wherein the connecting strap is irremovably attached to the anal or prostate stimulation device.

26. The male sexual stimulation apparatus as claimed in claim 15, wherein the connecting strap is detachably attached to the anal or prostate stimulation device.

27. The male sexual stimulation apparatus as claimed in claim 15, wherein the connecting strap has at least a nub or bulge near or adjacent to the base of the anal or prostate stimulation device.

28. The male sexual stimulation apparatus as claimed in claim 15, wherein the connecting strap has ridges or textures or contours on the upper surface adjacent to or near the base of the anal or prostate stimulation device.

29. The male sexual stimulation apparatus as claimed in claim 15, wherein the connecting strap is either elastic or flexible or stretchable or rigid.

30. The male sexual stimulation apparatus as claimed in claim 15, wherein the connecting strap has at least one holder for holding the male penile masturbation device at its distal end opposite to the anal or prostate stimulation device.

31. The male sexual stimulation apparatus as claimed in claim 30, wherein the holder is in the form of one or more closed and flexible or elastic rings or loops.

32. The male sexual stimulation apparatus as claimed in claim 30, wherein the holder is in the form of one or more adjustable straps.

33. The male sexual stimulation apparatus as claimed in claim 15, wherein means for vibration or actuation or thermal heating or electro-stimulation is/are provided to at least one of the components of the apparatus.

34. A method for connecting an anal plug or male masturbation device to a male penile masturbation device; wherein a rigid or flexible or elastic connecting strap is employed to connect them; and wherein one end of the connecting strap is attached to the anal or prostate stimulation device, whereas other end is of the connecting strap is used as holder for holding a male penile masturbation device.
